(19) United States
(12) Patent Application Publication
(10) Pub. No.: US 2026/0354511 A1
(43) Pub. Date: Nov. 12, 2026

(54) SYSTEM AND METHOD USING A WEARABLE MESH GOWN AND HAT WITH BUILT IN EYE PROTECTIONTO BE UTILIZED DURING THETREATMENT OF NEONATAL JAUNDICE

(71) Applicant: Cocoombs, LLC, Friendswood, TX (US)

(72) Inventors: Kelley Bergeson, Friendswood, TX (US); Melissa Thomas, El Lago, TX (US)

(21) Appl. No.: 19/529,250

(22) Filed: Feb. 4, 2026

Related U.S. Application Data
(60) Provisional application No. 63/758,070, filed on Feb. 13, 2025.

(51) Int. Cl. A41D 13/12; A42B 1/0181; A61N 5/06

(52) U.S. Cl. CPC A41D 13/1272; A42B 1/0181; A61N 5/0621; A41D 2300/33; A41D 2500/50; A61N 2005/0663

(57) ABSTRACT

A wearable neonatal phototherapy gown, having a tubular body formed from a stretchable, light-permeable mesh material, a flexible neck opening configured to stretch over a wearer's head, a closable lower end operable to encase the wearer's lower extremities, wherein the mesh material simultaneously permits therapeutic light transmission and creates a swaddling effect when the lower end is closed. Built-in eye protection on the skull cap comprised of a densely knitted fabric is aligned with the eye sockets and held in place by the cap. The cap material, excluding eyewear, may have a porosity selected to transmit therapeutic phototherapy and created by traditional knitting techniques.

### CROSS REFERENCE TO RELATED APPLICATIONS

This application claims priority to and the benefit of prior filed U.S. Provisional Patent Application Ser. No. 63/785,070, filed Feb. 13, 2025 hereby incorporated by reference herein.

### FIELD OF THE INVENTION

The present invention relates to a system and method that aids in the comfort during the treatment of Jaundice in infants, and more particularly to a System and Method Using a Wearable Mesh Gown and Hat with Built in Eye protection to be Utilized During the Treatment of Neonatal Jaundice. The present invention is intended to be disposable to increase accessibility in the hospital setting.

### BACKGROUND OF THE INVENTION

Jaundice and its need for treatment is universal as all infants throughout the world have the risk of developing jaundice that may require phototherapy treatment. Three in five infants in the newborn nursery will have jaundice from either a physiologic or pathologic source within the first week of life. An overall 10% of infants require phototherapy.

In general, a so-called flexible phototherapy treatment device is composed of a visible light source that emits at wavelengths corresponding to blue light. The blue light absorbed by bilirubin enables free bilirubin to be converted into water-soluble degradation products easily excreted in urine and stool that is harmless to humans.

Prior art solutions fail to properly cover the infant or are cumbersome using hook and loop fasteners or other straps to cover the infant. These systems take longer to put into place, fail to adequately comfort the infant, and result in less effective phototherapy. One such solution is the Bili Pod® created by Woombie. This product does not provide comfort to infants per its intent. The fabric does not conform to the infant's body, requires extensive use of cumbersome hook and loop fasteners and does not create a womb like environment. You also must use separate eye protection during phototherapy treatment.

Further, prior art devices are designed to accommodate the patient in a position resulting in areas that are not exposed to the light emitted by the light source. Consequently, these devices do not allow for uniform exposure over the whole patient's body. Treatment effectiveness is not therefore maximized by these devices.

Other body positioners, such as blankets, may be wrapped around the patient, and can accommodate an optical fiber-based pad with a view to phototherapy treatment, but this does not allow for the achievement of a high intensity treatment over long periods of time, for example 10 to 12 hours. These are also considered unsafe by the American Academy of Pediatrics safe sleep recommendations.

### BRIEF SUMMARY OF THE INVENTION

A primary advantage of a preferred embodiment of the invention is to provide a fitted sleeveless gown and cap with built-in eye protection set that is used during the treatment of phototherapy. The unique mesh design allows light treatment to penetrate the skin while creating a womb-like environment and providing a swaddling effect for safety and comfort.

Another advantage of the preferred embodiment of the invention is it addresses a need for comfort measures during phototherapy treatment.

Yet another advantage of the preferred embodiment of the invention is it resolves the frustrations inherent in neonatal care. It allows nurses to calm infants to keep them under the lights for effective treatment while also having the ability to obtain visualized focused assessments when necessary.

Another advantage is the all in one system includes the preferred embodiment with the benefits stated above, along with eye protection required during phototherapy treatment.

Yet another advantage is that the system may be disposable which makes it more accessible and decreases the hospital cost of laundering.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a perspective view of a mesh gown according to a preferred embodiment of the invention.

FIG. 2 is a front perspective view of a cap for placement over the infant head with reinforced stretchable hem according to a preferred embodiment of the invention.

FIG. 3A is a perspective view of an eye covering from the rear according to a preferred embodiment of the invention.

FIG. 3B is a perspective view of an eye covering according to a preferred embodiment of the invention.

FIG. 4 is a front perspective view of the foam insert for the eye cover according to a preferred embodiment of the invention.

FIG. 5 is a front perspective view of the eye cover with foam insert according to a preferred embodiment of the invention.

FIG. 6A is a front perspective view of an infant in the mesh gown, cap and built in eye cover with the bottom of the mesh gown open according to a preferred embodiment of the invention.

FIG. 6B is a front perspective view of an infant in the mesh gown, cap and built in eye cover with the bottom of the mesh gown closed according to a preferred embodiment of the invention.

FIG. 6C is a back perspective view of an infant in the mesh gown, cap and built in eye cover with the bottom of the mesh gown closed according to a preferred embodiment of the invention.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for later filed claims and as a representative basis for teaching one skilled in the art to employ the present invention in virtually any appropriately detailed system, structure or manner.

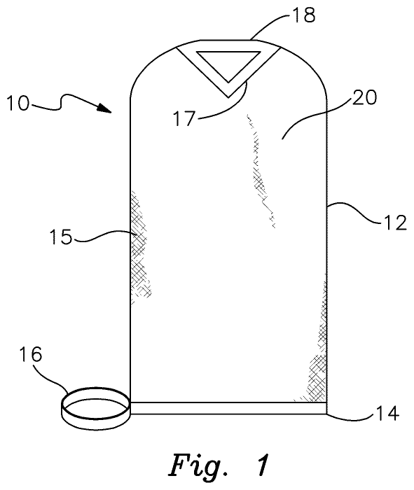

Turning now to FIG. 1 there is shown a front view of the wearable garment during the treatment of neonatal jaundice. Gown 10 is comprised of body 20 using mesh fabric 12 having porosity 15 of any of a variety of compositions described in more detail below. In a preferred embodiment, the mesh fabric is 81% Nylon and 19% Spandex. Body 20 may preferably be 27 inches in length but depending on use may of any of a variety of dimensions to accommodate the user's size. Gown 10 has soft V-neck opening 17 with collar material 18 that may be stretchable for placement over an infant's head. The neckline may optionally include an adjustable closure to ensure appropriate fitting depending upon infant size. This would be a closure with a hook and loop fastener style. Collar material may be approximately 0.5 inches in width and have an overall circumference of 12 inches with a 90-degree point in the front forming the V in V-neck opening 17.

Gown 10 is generally tubular or rectilinear of any of a variety of mesh fabric 12. The fabric may be composed of nylon fabric with spandex having small openings that permit the desired amount of light to pass through gown 10 to reach an infant. A preferred material is 81% nylon and 19% spandex, but the invention is not limited to this percentage of fabric blend and will work with a variety of materials. At the bottom of gown 10 is a drawstring 14 fabricated within or attached to body 20 having pull loop 16 for tightening of the bottom portion of body 20. Drawstring 14 may be 5 inches in diameter with an overall circumference of approximately 15 inches. Pull loop 16 may have a diameter of 3 inches. Any of a variety of dimensions may be employed to accommodate different sized infants or children.

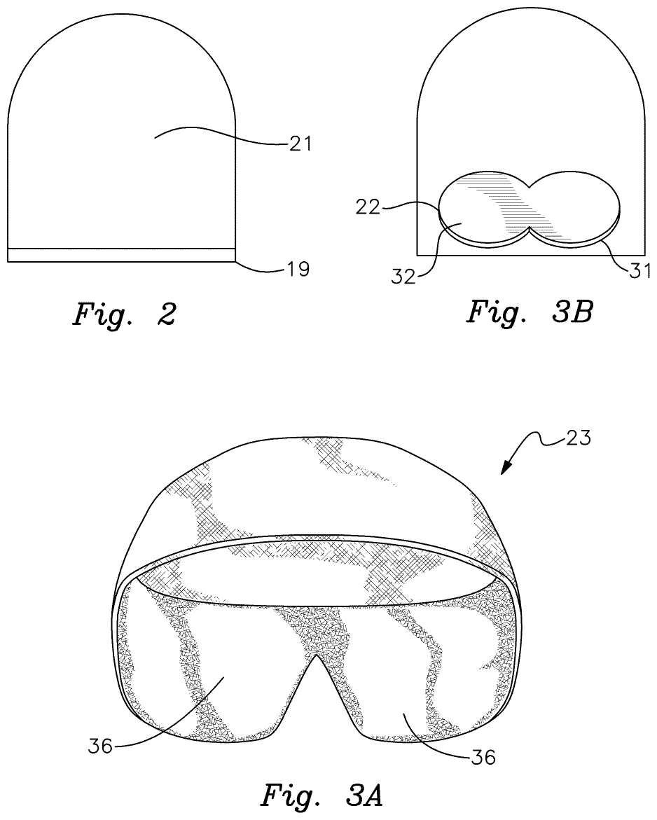

Turning to FIG. 2, there is shown cap 21 for placement over an infant's head during phototherapy with stretchable rim 19 for stable engagement to the head. In a preferred embodiment, cap 21 is generally shaped like a ski cap with a vertical dimension of approximately 6 inches and an opening bottom diameter of 5 inches for placement over the head. Rim 19 may be of any suitable stretchable material and be approximately 0.25 inches in width. The rim could potentially embody an adjustable closure for proper securement to the head. The closure could be a hook and loop fastener at the base of the head/neck.

FIG. 3A shows a perspective view from the rear orientation of skull cap 23 with integrated eyeflaps 36 positioned to cover the eyes when the skull cap 23 is placed over the head. When in position, eyeflaps 36 cover the eyes of an infant during the use of the system. Eyeflaps 36 may be knitted as a part of skull cap 23 and may have a tighter or reinforced area of knitting of higher density than skull cap 23. FIG. 3B is a side view of eye covering 22 behind see-through image of cap 21 for placement over the infant's eyes during phototherapy treatment. Eye cover 22 may be comprised of two connected generally circular pads 31 and 32 each pad extending about 2 inches in width and having a vertical length for each eye pad of about 1.75 inches. Whether cap 21 or skull cap 23, either may be employed with gown 10 during the treatment of neonatal jaundice

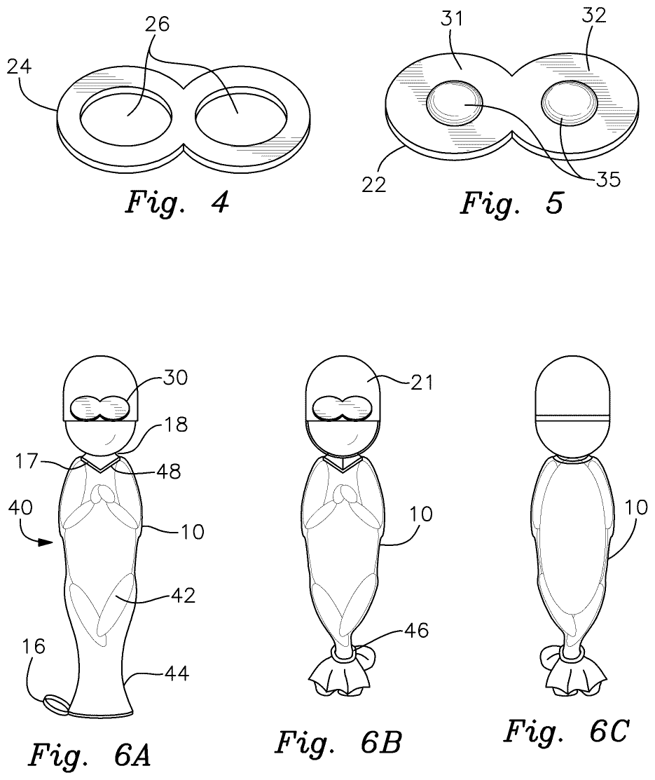

FIG. 4 is a perspective view of an optional foam interior 24 of eye cover 22 with openings 26 for each circular eyepads 31 and 32 in eye cover 22. FIG. 5 is a perspective view of an optional eye cover 22 fully assembled with circular eyepads 31 and 32 adhered to foam interior 24 for placement over an infant's eyes. In a preferred embodiment, eyepads 31 and 32 each have an interior raised region 35 for each eye to provide additional space over the eye socket and eye lashes and for comfort. The eye coverings 22 may be composed of a dense hypoallergenic foam material.

FIG. 6A is a diagrammatic depiction of infant 40 within gown 10. Before utilizing light therapy, gown 10 is placed over the head of infant 40 through bottom opening 44 all the way through neck opening 17 and collar 18 at neck region 48 with legs 42 fully encased in gown 10. Collar 18 has a flexible trim to permit stretching over the infant's head for secure placement around the neck region 48. Once in place as shown in FIG. 6A, pull loop 16 is drawn about opening 44 to create tightened loop region 46 for secure placement about the lower extremities of infant 40. FIGS. 6B and 6C show a front and rear view of gown 10 fully cinched about the infant 40. Eye cover 30 (shown in FIG. 6A) is emplaced under cap 21 and stably secured thereto the infant's face and over the eye sockets. Alternatively, skull cap 23 may be placed over the head of the infant and cover the eye regions with eyeflaps 36 as previously described. The method for creating reinforced eyeflaps 36 in skull cap 23 and attaching it as part of skull cap 23 by various knitting processes is well known in the art.

In operation, the fully covered infant may be exposed to a variety of light therapeutic systems for the treatment of jaundice or other neonatal conditions. In a preferred embodiment of the invention, porosity 15 of body 20 may be variable depending on the fabric used. In one preferred embodiment, the gown may be composed of a nylon fabric with spandex having small openings that permit the appropriate amount of light to impact the infant's body. The fabric may be composed of other fabrics so long as the fabric permits an appropriate amount of light to the infant. A stretchable fabric permits wider use among different sized infants and a fine tuning of the porosity. Although yarn configuration can be any variety of means, several preferred embodiments are discussed herein

In a preferred embodiment of the invention, two knitted textile items can be used: an infant cap and a body wrap. Both items are produced using fully knitted, complete garments formed directly on a knitting machine. In a preferred embodiment, eye protection may be created by a densely knitted fabric for eyeflaps on the skull cap for protection without additional eye covers. No cutting, sewing, or post-knit assembly operations are required. The technical development focused on yarn selection, stitch structure, and zoned knitting techniques to control elasticity, fit, and fabric behavior.

### Yarn Configuration

Common Yarns Used (Cap and Body Wrap) for both the infant cap and body wrap utilize the following yarns: 2/70/34 stretch nylon yarn. This yarn forms the primary structure of both garments and is used throughout the majority of the knitted fabric.

120-4/12-70/24 yarn. This yarn is used in combination with the 2/70/34 stretch nylon yarn to introduce controlled elasticity, allowing the garments to gently conform to the body through fabric behavior rather than rigid compression.

For the cap, additional Yarn Used 140 denier DC elastic yarn, wrapped by two 1/70/34 yarns. This wrapped elastic configuration may be used in the infant cap, in localized functional regions. The wrapped construction provides elastic recovery while maintaining a soft surface suitable for infant contact.

The 140d DC elastic yarn is not preferred for use in the body wrap.

### Knitted Structures and Zoning

### Mesh Construction

The primary knit structure used in both the infant cap and body wrap is a mesh stitch pattern. This structure was selected to increase openness of the fabric while maintaining garment integrity and positioning. The mesh regions are formed using a programmed knitted stitch structure that increases fabric openness through controlled stitch variation.

The mesh structure provides localized porosity while maintaining sufficient structural integrity to preserve garment shape and positioning. The degree of openness is determined by stitch configuration and yarn interaction rather than by post-knit perforation or cutting. The mesh stitch structure interacts with the selected yarns to influence elasticity, drape, and dimensional behavior of the garment. The mesh structure forms the majority of the garment surface in both items.

### Jersey Knit Region (Body Wrap)

In the body wrap, the neck area is knitted using a jersey stitch structure, distinct from the mesh used in the remainder of the garment. This structural change is achieved through stitch programming during knitting and does not require additional assembly.

### 3.3 Spacer Fabric (Cap Only)

A spacer-like knitted fabric is used exclusively in the eye protection region of the infant cap. This structure is formed by combining 2/70/34 stretch nylon yarn and 140d DC elastic yarn (wrapped). The spacer region creates a three-dimensional fabric zone and includes a knitted notch that allows for nose exposure, similar in function to eyeglasses. The spacer fabric is not used in any other area of the cap or body wrap.

### Garment Formation and Finishing

In a preferred embodiment, both the infant cap and body wrap are knit complete, meaning: The full garment shape is formed directly on the knitting machine, edges and openings are integrated during knitting, and there is no cutting, sewing, or post-knit finishing operations are required other than traditional wash, dry and steam.

Garment geometry and fit are controlled through stitch structure, yarn selection, and programmed knitting parameters.

### Technical Considerations

Changes in yarn construction, yarn combination, or stitch structure inherently affect fabric behavior, including elasticity, dimensional stability, and finished geometry. These effects occur even when the overall garment shape and knit program logic remain unchanged. Certain geometric and functional characteristics arise from the interaction of yarn type and stitch structure rather than from explicit geometric programming alone. These variations do not affect the overall effectiveness of the preferred embodiments of the invention.

While the invention has been described in connection with preferred embodiments, it is not intended to limit the scope of the invention to the particular form set forth, but on the contrary, it is intended to cover such alternatives, modifications, and equivalents as may be included within the spirit and scope of the invention as defined by the later issued claims.

1. A wearable mesh gown and hat with built in eye protection to be used during phototherapy treatment, comprising:
a wearable, sleeveless gown formed from a stretchable mesh fabric having a porosity selected to transmit therapeutic phototherapy light through substantially the entire surface area of the gown;
a neck opening configured to pass over a wearer's head and rest at a neck region;
a lower portion of the gown defining an open end;
a selectively cinchable closure integrated at the open end and operable to encase a infant's lower extremities while maintaining tension in the mesh fabric to create a swaddling effect; and
a cap configured to be worn on the wearer's head during phototherapy; and
eye-protection retained against the infant's eyes, wherein the system permits continuous phototherapy exposure while maintaining neonatal comfort and reducing movement.

2. The system of claim 1, wherein the mesh fabric comprises a nylon and spandex blend configured to elastically conform to a wearer's body.

3. The system of claim 1, wherein the porosity of the mesh fabric permits blue-light wavelengths used in bilirubin reduction to pass through the gown without removal of the garment.

4. The system of claim 1, wherein the selectively cinchable closure comprises a drawstring positioned within a hem at the open end of the gown.

5. The system of claim 4, wherein the drawstring includes a pull loop sized for manual tightening by a caregiver.

6. The system of claim 1, wherein the gown remains fully wearable during the entirety of a phototherapy treatment session.

7. The system of claim 1, wherein the cap includes a stretchable rim configured to maintain position without external fasteners.

8. The system of claim 1, further comprising a skull cap with integrated eye covering of a more densely knitted fabric over the eyes for protection.

9. The system of claim 1, wherein the gown provides uniform phototherapy exposure without requiring repositioning of the infant.

10. A wearable neonatal phototherapy gown, comprising:
a tubular body formed from a stretchable, light-permeable mesh material;
a flexible neck opening configured to stretch over a infant's head; and
a closable lower end operable to encase the infant's lower extremities,
wherein the mesh material simultaneously permits therapeutic light transmission and creates a swaddling effect when the lower end is closed.

11. The gown of claim 10, wherein the tubular body is sleeveless to maximize exposed skin surface area.

12. The gown of claim 10, wherein the lower end includes an integrated loop closure.

13. The gown of claim 10, wherein elastic tension of the mesh fabric provides calming compression without restricting circulation.

14. The gown of claim 10, wherein the gown is sized to accommodate a range of neonatal body sizes without adjustment to the mesh structure.

15. A method of providing comfort during the treatment of neonatal jaundice, comprising:
placing an infant into a wearable mesh gown that permits phototherapy light to pass through the gown to the infant's skin;
cinching a lower end of the gown to encase the infant's lower extremities and create a swaddling effect;
positioning a built-in eye-protection assembly over the infant's eyes using a wearable cap; and
exposing the infant to phototherapy light while the infant remains continuously swaddled within the gown.

16. The method of claim 15, wherein the phototherapy light comprises blue-light wavelengths effective for bilirubin reduction.

17. The method of claim 15, wherein the gown could remain on the infant throughout an entire phototherapy session.

18. The method of claim 15, wherein the infant is comforted during phototherapy.

19. The method of claim 15, wherein the method complies with neonatal safe-sleep guidelines by eliminating loose blankets or external restraints.

20. The method of claim 15 further comprising the placement of a skull cap with integrated eyeflaps during the phototherapy session.
